(19) United States
(12) Patent Application Publication
(10) Pub. No.: US 2024/0148634 A1
(43) Pub. Date: May 9, 2024

(54) A TISSUE FILLER COMPRISING HYALURONIC ACID GEL AND A PRODUCTION METHOD THEREOF

(71) Applicant: HACETTEPE UNIVERSITESI REKTORLUK, Cankaya/ Ankara (TR)

(72) Inventor: Halil Murat AYDIN, Ankara (TR)

(73) Assignee: HACETTEPE UNIVERSITESI REKTORLUK, Cankaya/Ankara (TR)

(21) Appl. No.: 18/272,652

(22) PCT Filed: Jan. 25, 2022
(86) PCT No.: PCT/TR2022/050060

§ 371 Date: Jul. 17, 2023

(30) Foreign Application Priority Data
Jan. 25, 2021 (TR) 2021/01065

(51) Int. Cl. A61K 8/73; A61K 8/365; A61Q 19/08

(52) U.S. Cl. CPC A61K 8/735; A61K 8/365; A61Q 19/08; A61K 2800/5922; A61K 2800/882; A61K 2800/91

(57) ABSTRACT

The present invention relates to a tissue filler which comprises two different phases, namely cross-linked and non-cross-linked; wherein tricarboxylic acid is included within non-cross-linked phase; which is made of hyaluronic acid gel and a production method of this filler.

### TECHNICAL FIELD

The present invention relates to a tissue filler which comprises two different phases, namely cross-linked and non-cross-linked; wherein tricarboxylic acid is included within non-cross-linked phase; which is made of hyaluronic acid gel and a production method of this filler.

### BACKGROUND OF THE INVENTION

Effects of aging occur on the face due to the effects of gravity, muscular movements and sunbeams in the course of time. These effects appear in the form of wrinkles and lines. Soft tissue fillers are frequently applied in the treatment of such effects associated with aging. Hyaluronic acid (HA)-based gels are one of these fillers. HA is a water-soluble natural polysaccharide. It exhibits high-level of biocompatibility and its allergic reaction potential is minimum. Because of its water retention characteristic as much as hundreds of times of its weight, it is volumizing and commonly used in filler applications. The fact that they decrease within the soft tissue in a very short time when they are implanted in the body is the disadvantage of hyaluronic acid-based fillers.

Many dermal fillers which are currently used are two-phase hyaluronic acid systems that are distributed, cross-linked and turned into a particle form within non-cross-linked hyaluronic acid gel. However, even though these fillers can create a good volume effect, their properties of binding together are low because they are made of two different phases. Cohesion can be briefly defined as a sign for property of remaining together of a get in this form. An obtained product is required to exhibit cohesive property in terms of efficiency of gel both in application and after application.

In addition to these, a product with different rheological properties is needed for many different areas such as lip, forehead, nose, jawline, fine superficial wrinkles especially in intradermal applications. A cross-linker is used at different levels in order to reach a high elasticity value desired in these products. Butanediol diglycidyl ether (BDDE), which is particularly preferred as cross-linker, is highly toxic. A higher elasticity can be obtained by increasing the amount of BDDE and this product is used in areas wherein more volume is needed. For example, 5 different cross-linker ratios (amounts) are used while the phase of cross-linked hyaluronic acid is produced for a product portfolio that will consist of 5 series; high BDDE is used in areas requiring particularly high modulus of elasticity; durations of dialysis needed to remove this BDDE—which should be included in the final product in an amount less than 2 ppm in accordance with the U.S. Food and Drug Administration (FDA)—from the structure get quite longer (from 2 days to 7 days).

Therefore, there is need for a new tissue filler that will overcome all these problems stated above and a production method thereof.

The United States patent document no. US2020086009, an application in the state of the art, discloses injectable soft tissue fillers and more specifically hyaluronic acid-based (HA) fillers. In the said invention, the HA-based component is a gel comprising cross-linked particles in a fluidic medium of free HA. In one embodiment of the invention, the HA component is for example a cohesive, hydrated gel and the said HA component is a cross-linked gel having no greater than 1% to 10% free HA. In the said invention, the HA is linked with butanediol diglycidyl ether and the said HA component has a degree of crosslinking of less than about 5%. The cross-linked HA gel is neutralized for example by adding an aqueous solution comprising an acid such as HCL to obtain the desired cohesivity and the cross-linked HA gel is then swelled in a phosphate buffered saline solution for a sufficient time. After the addition of the HCl, it is ensured that the HA gel is homogenized to have a desired consistency and stability. During the homogenization, transaction of mixing the gels is carried out in a controlled way.

### SUMMARY OF THE INVENTION

An objective of the present invention is to realize a tissue filler which comprises two different phases, namely cross-linked and non-cross-linked; wherein tricarboxylic acid is included within non-cross-linked phase; which is made of hyaluronic acid gel and a production method of this filler.

Another objective of the present invention is to realize a production method of tissue filler with improved cohesion properties by using a non-toxic component.

Another objective of the present invention is to realize a tissue filler which enables to adjust cohesion force by changing the elasticity value of a non-cross-linked hyaluronic acid phase upon the ratio of tricarboxylic acid in its content is changed and a production method thereof.

Another objective of the present invention is to realize a tissue filler with a biphasic cohesive gel content which provides different rheological properties for different applications by formulation of both hyaluronic acid phases, namely cross-linked and non-cross-linked, by using hyaluronic acid at low, medium and high molecular weight and a production method thereof.

Another objective of the present invention is to realize a tissue filler wherein the amount of cross-link can be can be changed in the cross-linked phase a production method thereof.

### DETAILED DESCRIPTION OF THE INVENTION

The inventive tissue filler is a hyaluronic acid gel which comprises two different phases, namely cross-linked and non-cross-linked; wherein non-toxic tricarboxylic acid is included in the non-cross-linked phase and the rheological properties of a final product range can be adjusted by changing the ratio of tricarboxylic acid within the non-cross-linked phase, instead of modification of the cross-linked phase.

The cross-linked phase of the inventive tissue filler comprises hyaluronic acid and butanediol diglycidyl ether used as the cross-linker.

The non-cross-linked phase of the inventive tissue filler comprises hyaluronic acid and tricarboxylic acid. At least 0.5% tricarboxylic acid by weight is included within the non-cross-linked phase of the inventive tissue filler. In one embodiment of the invention, the tricarboxylic acid in the non-cross-linked phase of the tissue filler is citric acid.

### Method

The inventive production method of tissue filler comprises steps of:
obtaining a cross-linked phase by adding a cross-linking component into hyaluronic acid;
obtaining a non-cross-linked phase by adding tricarboxylic acid into hyaluronic acid; and
obtaining a tissue filler by combining a cross-linked phase and a non-cross-linked phase.

At the step of obtaining a cross-linked phase of the inventive production method of tissue filler, a cross-linker is added into hyaluronic acid in the form of butanediol diglycidyl ether. A cross-linking reaction is chemically carried out between hyaluronic acid and butanediol diglycidyl ether in a basic medium. An unreacted cross-linker is removed following the cross-linking. Then, the gel occurring by the cross-linking of butanediol diglycidyl ether with hyaluronic acid is buffered. A cross-linked phase is obtained by reducing the buffered gel into a particle form.

At the step of obtaining a non-cross-linked phase of the inventive production method of tissue filler, at least 0.5% citric acid by weight is added into hyaluronic acid as tricarboxylic acid. A non-cross-linked phase with a cohesive property which enables to keep two different phases together is obtained by combining hyaluronic acid and citric acid in a phosphate buffer without carrying out any chemical reaction.

At the step of combining a cross-linked phase and a non-cross-linked phase of the inventive production method of tissue filler, a cross-linked phase and a non-cross-linked phase are combined and homogenized by being mixed mechanically for 24 hours and a tissue filler is obtained.

The tissue filler produced by the inventive method is injected into soft tissues in the body for the purpose of treatment of wrinkles and lines associated with aging. The tissue filler can also be used in ophthalmic applications in addition to being used for preventing wrinkle formation, intraarticularly in the form of viscosupplementation gels or for preventing postoperative adhesion or as tissue scaffold.

Secondary interactions and/or plasticizing behavior emerge among hyaluronic acid chains, by means of citric acid that is included in non-cross-linked phase of the tissue filler produced with the inventive method. In addition, cohesion property occurs by means of citric acid and it becomes possible to enhance interaction of hyaluronic acid chains and to adjust rheological properties such as elasticity and loss value easily, by means of the cohesion property. It is ensured to keep particles of two phases, which are cross-linked and non-cross-linked in the tissue filler, together through the use of citric acid. The characteristic of cross-linked particles to be injectable into body tissue homogeneously by means of citric acid is improved and it is ensured that particles within a filler can be applied to a dermal area without dispersing and easily in dispersion of filler to an application area by an operator. It is facilitated to obtain different products that can provide rheological properties suitable for different application points such as eye, lip, deep wrinkle by changing the amount of tricarboxylic acid included within non-cross-linked phase in the tissue filler.

In the inventive tissue filler, extremely high increases are seen in cohesion properties of the non-cross-linked gel by addition of a non-toxic tricarboxylic acid, preferably a citric acid, into a non-cross-linked hyaluronic acid phase in certain ratios. This interaction which considerably results from physical interactions has a potential to bring a an important characteristic to fillers wherein cross-linked hyaluronic acid particles will be dispersed. By changing the ratio of tricarboxylic acid to be applied to a tissue filler, it is possible to change the elasticity and loss values of a non-cross-linked hyaluronic acid phase and to adjust the cohesion forces. Both hyaluronic acid phases, namely cross-linked and non-cross-linked, in a tissue filler can be formulated by using hyaluronic acid at low, medium and high molecular weight and thus biphasic cohesive gels providing different rheological properties for different indications can be produced. Similarly, the amount of cross-link can be changed in a cross-linked fraction. Antioxidants, antiseptics, anti-inflammatories, local anesthetics and mixtures thereof can be added into the tissue filler. In addition, the tissue filler can comprise chondroitin sulfate or other polysaccharides. The tissue filler can be injected into body by means of needles with suitable measuring diameter. The tissue filler can be sterilized by means of moist heat. The tissue filler enables to acquire the rheological properties necessary for different applications by changing the non-cross-linked phase comprising tricarboxylic acid in a short time without needing extra chemical reactions, upon the cross-linked phase in thereof is used as stock.

Within these basic concepts; it is possible to develop various embodiments of the inventive “Tissue Filler Comprising Hyaluronic Acid Gel and a Production Method Thereof (1)”; the invention cannot be limited to examples disclosed herein and it is essentially according to claims.

1. A tissue filler which is a hyaluronic acid gel that comprises two different phases, namely cross-linked and non-cross-linked; wherein non-toxic tricarboxylic acid is included in the non-cross-linked phase and the rheological properties of a final product range can be adjusted by changing the ratio of tricarboxylic acid within the non-cross-linked phase, instead of modification of the cross-linked phase.

2. A tissue filler according to claim 1, characterized by the cross-linked phase which comprises hyaluronic acid and butanediol diglycidyl ether used as the cross-linker.

3. A tissue filler according to claim 1, characterized by the non-cross-linked phase which comprises hyaluronic acid and tricarboxylic acid.

4. A tissue filler according to claim 1, characterized by the non-cross-linked phase wherein at least 0.5% tricarboxylic acid by weight is included.

5. A tissue filler according to claim 1, characterized by the non-cross-linked phase which comprises citric acid as tricarboxylic acid.

6. A method of obtaining tissue filler; characterized by steps of:
obtaining a cross-linked phase by adding a cross-linking component into hyaluronic acid;
obtaining a non-cross-linked phase by adding tricarboxylic acid into hyaluronic acid; and
obtaining a tissue filler by combining a cross-linked phase and a non-cross-linked phase.

7. A method according to claim 6, characterized in that a cross-linker is added into hyaluronic acid in the form of butanediol diglycidyl ether, at the step of obtaining a cross-linked phase.

8. A method according to claim 7, characterized in that a cross-linking reaction is chemically carried out between hyaluronic acid and butanediol diglycidyl ether in a basic medium.

9. A method according to claim 8, characterized in that an unreacted cross-linker is removed following the cross-linking.

10. A method according to claim 9, characterized in that the gel occurring by the cross-linking of butanediol diglycidyl ether with hyaluronic acid is buffered.

11. A method according to claim 10, characterized in that a cross-linked phase is obtained by reducing the buffered gel into a particle form.

12. A method according to claim 6, characterized in that at least 0.5% citric acid by weight is added into hyaluronic acid as tricarboxylic acid, at the step of obtaining a non-cross-linked phase.

13. A method according to claim 12, characterized in that a non-cross-linked phase with a cohesive property which enables to keep two different phases together is obtained by combining hyaluronic acid and citric acid in a phosphate buffer without carrying out any chemical reaction.

14. A method according to claim 6, characterized in that a cross-linked phase and a non-cross-linked phase are combined and homogenized by being mixed mechanically for 24 hours and a tissue filler is obtained, at the step of combining a cross-linked phase and a non-cross-linked phase.

15. A tissue filler according to claim 1, which is used in soft tissues in the body for the purpose of treatment of wrinkles and lines associated with aging.

16. A tissue filler according to claim 1, which is also used in ophthalmic applications, intraarticularly in the form of viscosupplementation gels or for preventing postoperative adhesion or as tissue scaffold.
